(19) United States
(12) Patent Application Publication
(10) Pub. No.: US 2011/0244503 A1
(43) Pub. Date: Oct. 6, 2011

(54) System and Method for Anti-Cancer Drug Candidate Evaluation

(76) Inventors: Mathieu Perrée, Verdun (CA); Allan E. Hallquist, Morning View, KY (US)

(21) Appl. No.: 12/750,962

(22) Filed: Mar. 31, 2010

(51) Int. Cl. C12Q 1/02

(52) U.S. Cl. 435/29

(57) ABSTRACT

The disclosure provides a method of evaluating the ability of an anti-cancer drug candidate to induce apoptosis in a known cancer cell line by placing a single-cell suspension of a known cancer cell line in a well of a plate, adding at least one drug candidate to the well in an amount sufficient to achieve a target drug candidate concentration, measuring the optical density at selected time intervals for a selected duration of time, determining a kinetic units value from the optical density and time measurements, and correlating the kinetic units value with an ability of the anti-cancer drug candidate to induce apoptosis in the cancer cell line if the kinetic units value is positive. A similar method may be used to evaluate the ability of an anti-cancer drug candidate to induce apoptosis in a cancer type.

### TECHNICAL FIELD

The current invention relates to use of a spectrophotometric assay to evaluate the ability of anti-cancer drug candidates to induce apoptosis in cancer cells.

### BACKGROUND

Cell death may occur in a variety of manners, but successful anti-cancer drugs tend to cause death of cancer cells by the very specific process of apoptosis. Apoptosis is a mechanism by which a cell disassembles and packages itself for orderly disposal by the body. Apoptosis is commonly used by the body to discard cells when they are no longer needed, are too old, or have become damaged or diseased. In fact, some cells with dangerous mutations that might lead to cancer and even some early-stage cancerous cells may undergo apoptosis as a result of natural processes.

During apoptosis, the cell cuts and stores DNA, condenses the nucleus, discards excess water, and undergoes various changes to the cell membrane, such as blebbing, the formation of irregular bulges in the cell membrane. (See FIG. 1.) Apoptosis generally occurs after one of several triggers sends a signal to the cell that it should undergo apoptosis. In many cancer cells, this message system does not work correctly because the cell cannot detect the trigger, fails to send a signal properly after the trigger is received, or fails to act on the signal, or the cell may even have combinations of these problems. The overall effect is a resistance to undergoing apoptosis in some cancer cells.

Cancer, as used herein, includes epithelial malignancies, leukemia, lymphomas and mesenchymal malignancies. Many effective cancer drugs can induce a cancer cell to undergo apoptosis despite its resistance to the process. Accordingly, there is a need to detect whether a particular drug candidate can cause apoptosis in various types of cancer cells and also to determine the drug candidate's effectiveness as compared to other drugs or drug candidates.

The MiCK assay, described in U.S. Pat. No. 6,077,684 and U.S. Pat. No. 6,258,553 is currently used to detect whether cancer cells from a patient undergo apoptosis in response to a particular drug known to be effective against one or more types of cancer. In the MiCK assay cancer cells from a patient are placed in a suspension of a given concentration of single cells or small cell clusters and allowed to adjust to conditions in multiple wells of a microtiter plate. Control solutions or solutions with various concentrations of known anti-cancer drugs, typically those drugs recommended for the patient's cancer type, are introduced into the wells with one test sample per well. The optical density of each well is then measured periodically, typically every few minutes, for a period of typically a few days. As a cell undergoes apoptosis-related blebbing, its optical density increases in a linear fashion. If the cell does not undergo apoptosis or dies from other causes, its optical density does not change in this manner. Thus, if a plot of optical density (OD) v. time for a well yields a straight line curve having a positive slope over the time interval (see FIG. 2), then the anti-cancer drug in that well induces apoptosis of the patient's cancer cells and might be a suitable therapy for that patient. OD v. time data may also be used to calculate kinetic units, which similarly correlate with the suitability of a therapy for the patient.

Although the MiCK assay has been used to detect the effects of known anti-cancer drugs on a particular patient's cancer cells, there remains a need to develop variations of the assay able to explore and evaluate other types of apoptosis-related cell/chemical interactions.

### SUMMARY

The present disclosure provides a method of evaluating the ability of an anti-cancer drug candidate to induce apoptosis in a known cancer cell line. The method may include placing a single-cell suspension of viable cancer cells from a known cancer cell line in at least one well of a plate able to be read by a spectrophotometer, wherein the cancer cells are in a concentration sufficient to form a monolayer of cells on the bottom of the well, adding at least one drug candidate to the well in an amount sufficient to achieve a target drug candidate concentration, measuring the optical density of the well at a wavelength of approximately 600 nm using a spectrophotometer at selected time intervals for a selected duration of time, determining a kinetic units value from the optical density and time measurements, and correlating the kinetic units value with an ability of the anti-cancer drug candidate to induce apoptosis in the cancer cell line if the kinetic units value is positive, or an inability of the anti-cancer drug candidate to induce apoptosis in the cancer cell line if the kinetic units value is not positive.

According to a further embodiment, a similar method may be used to evaluate the ability of an anti-cancer drug candidate to induce apoptosis in a cancer type, where the known cancer cell line used in the assay is of the cancer cell type.

According to more specific embodiments, correlating may include correlating the kinetic units value with an ability of the anti-cancer drug candidate to induce apoptosis in the cancer cell type if the kinetic units value is greater than 1.5, 2, or 3, and an inability of the anti-cancer drug candidate to induce apoptosis in the cancer cell type if the kinetic units value is less than 1.5, 2, or 3, respectively. Correlating may also include correlating the kinetic units value with induction of spontaneous cell death or necrosis by the anti-cancer drug candidate if the kinetic units value is negative.

According to further specific embodiments, the cancer cells may be in a concentration of between 2×10⁵ and 1×10⁶ cells/mL. The cancer cells may be in an exponential or a non-exponential growth phase. In a specific embodiment, particularly when the cancer cells are from a cancer cell line they may be in an exponential growth phase.

According to other specific embodiments, at least one additional drug candidate may be added to the well in an amount sufficient to achieve an additional target drug candidate concentration.

According to further embodiments, The cancer cells may be placed in multiple wells of the plate and each well may have a different target drug candidate concentration. For example, the target drug candidate concentration may be between 0.01 and 10,000 μM.

According to additional embodiments, the selected time intervals may be 5 to 10 minutes. The duration of time may be between 12 hours and 120 hours.

The following abbreviations and terms are used commonly throughout this Specification:

OD—optical density.
MiCK—microculture kinetic.

### BRIEF DESCRIPTION OF THE DRAWINGS

A more complete understanding of the present embodiments and advantages thereof may be acquired by referring to the following description taken in conjunction with the accompanying drawings.

FIG. 1 shows a prior art photomicrograph of a cell. FIG. 1A shows the cell prior to apoptosis. FIG. 1B shows the cell during apoptosis when blebbing is occurring. FIG. 1C shows a cell when apoptosis is complete or nearly complete.

FIG. 2 is a prior art graph showing an example plot of time versus optical density (OD) during a MiCK assay in which an anti-cancer drug induces apoptosis in the cancer cells tested.

FIG. 3 is a graph showing representative curves for induction of apoptosis, drug resistance, and control cells without drug in a MiCK assay. The curve labeled “B12” shows data representative of cells in which the drug induces apoptosis. The curve labeled “F3” shows data representative of cells that are resistant to the drug. The curve labeled “G5” shows data representative of control cells that did not receive any drug.

FIG. 4 is a graph showing representative data for induction of apoptosis or necrosis in a MiCK assay. The curve labeled “D2” shows data representative of cells in which the drug induces apoptosis. The curve labeled “D7” shows data representative of cells in which the drug induces necrosis or which otherwise undergo necrosis during the course of the assay.

FIG. 5 is a graph showing representative data for general non-drug-induced cell death in a MiCK assay. The curve labeled “C4” shows data representative of spontaneous cell death during the course of the assay.

FIG. 6 is a graph showing representative date for the evaluation of the response of known cell lines corresponding to different cancer types to Idarubicin.

FIG. 7 is a graph showing representative data for the evaluation of the response of the known CAOV-3 ovarian cancer cell line to different chemotherapeutic agents.

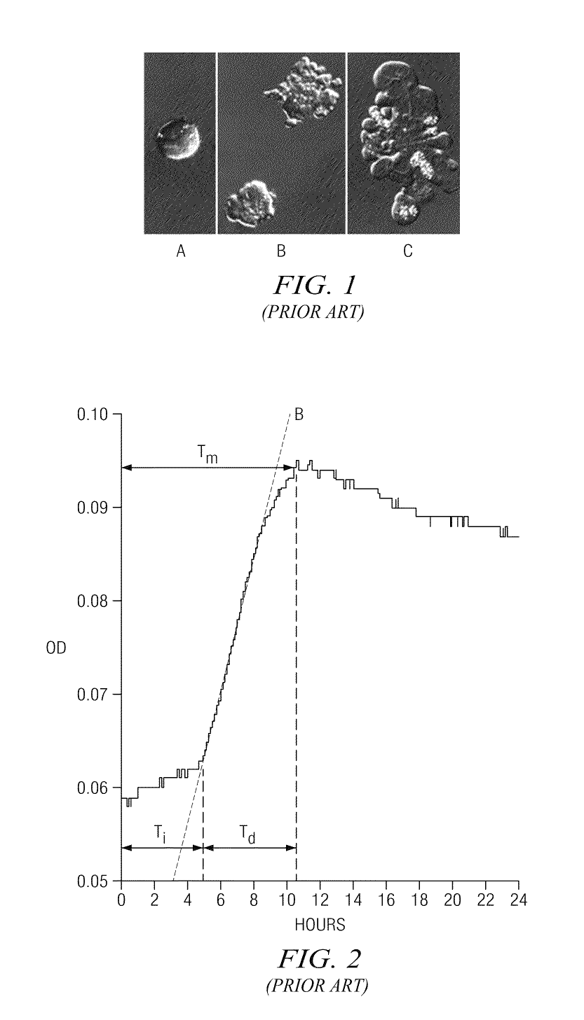

### DETAILED DESCRIPTION

The disclosure relates to evaluation of anti-cancer drug candidates' effectiveness in causing apoptosis in cancer cells using a spectrophotometric assay to measure optical density (OD) over a period of time. In one embodiment, the disclosure includes a method of evaluating such anti-cancer drug candidates by applying the drug candidates to cancer cells in an assay similar to the microculture kinetic (MiCK) assay as disclosed in U.S. Pat. No. 6,077,684 and U.S. Pat. No. 6,258,553, both incorporated by reference herein.

### Assay

According to one specific embodiment, the assay may proceed by selecting an anti-cancer drug candidate and selecting at least one known cancer cell type on which to test the drug. The cancer cells may be suspended as a single-cell suspension in culture medium, such as RPMI. As used herein, a “single cell suspension” is a suspension of one or more cells in a liquid in which the cells are separated as individuals or in clumps of 10 cells or fewer. The culture medium may contain other components, such as fetal-bovine serum or components specifically required by the cancer cells. These components may be limited to those necessary to sustain the cells for the duration of the assay, typically at least 24 hours and not longer than 120 hours.

Suspended cells may be tested by placing samples in wells of a spectrophotometric plate. The cells may be suspended at any concentration such that during the spectrophotometric measurements of OD, the beam of the plate reader normally passes through only one cell layer at a time. For most cells a concentration of between 2×10⁵ and 1×10⁶ cells/mL may be used. Concentration may be increased for small cells and decreased for large cells. To more precisely determine the appropriate cell concentration, the volume of cell suspension to be used in drug candidate test samples may be added to at least one concentration test well of the plate. If the well will be prefilled with additional medium during testing of the drug candidates, then the concentration test well may similarly be prefilled with additional medium. After the concentration test well is filled, the plate may be centrifuged (e.g. for 2 min at 500 RPM) to settle the cells on the bottom of the well. If the cell concentration is appropriate for the assay, the cells should form a monolayer without overlapping. Cell concentration may be adjusted as appropriate until this result is achieved. Multiple concentrations of cells may be tested at one time using different concentration test wells.

According to embodiments where the cells may grow significantly overnight or during another period of time between placement of the cells in the plate and commencement of the drug candidate assay, the cell concentration may be adjusted to initially achieve less than a monolayer to allow for growth such that sufficient cells for a monolayer will be present when the drug candidate assay commences.

The cancer cells may be in an exponential or a non-exponential growth phase. In a specific embodiment, particularly when the cancer cells are from a cancer cell line they may be in an exponential growth phase.

After the appropriate cell concentration has been determined, the drug-candidate assay may proceed by filling test and control wells in the plate with an appropriate volume of medium and an appropriate number of cells. In other embodiments the well may be partially pre-filled with medium alone.

After filling, the cells may be allowed to adjust to the plate conditions for a set period of time, such as at least 12 hours, at least 16 hours, at least 24 hours, or 12-16 hours, 12-24 hours, or 16-24 hours. An adjustment period may be omitted for certain cell types, such as leukemia/lymphoma cell lines or other cell types normally present as individual cells. The adjustment period is typically short enough such that the cells do not experience significant growth during the time. The adjustment period may vary depending on the type of cancer cells used in the drug candidate assay. Adjustment may take place under conditions suitable to keep the cells alive and healthy. For example, the plate may be placed in a humidified incubator at 37° C. under 5% CO₂ atmosphere. For some cell types, particularly cell types that do not undergo an adjustment period, such as leukemia or lymphoma cell lines, the plate may be centrifuged (e.g. for 2 minutes at 500 RPM) to settle the cells on the bottom of the wells.

The drug candidate and any control drugs or other control samples may be added to the wells after the adjustment period. Typically the drug candidate will be added in a small volume of medium or other liquid as compared to the total volume of liquid in the well. For example, the volume of drug added may be less than 10% of the total volume of liquid in the well. Drug candidates may be added in multiple dilutions to allow determination of any concentration effects. Although many drug candidates may be water-soluble, drug candidates that are not readily soluble in water may also be tested. Such candidates may be mixed with any appropriate carrier. Such candidates may preferably be mixed with carriers anticipated for actual clinical use. Viscous drug candidates may require substantial dilution in order to be tested. Drug candidates with a strong color may benefit from monitoring of OD in test wells containing only the drug candidate and subtraction of this OD from measurements for the test sample wells.

After addition of the drug candidate, the cells may be allowed another short period of adjustment, for example of 15 minutes or 30 minutes. The cells may be placed under conditions suitable to keep the cells alive and healthy. For example, the plate may be placed in a humidified incubator at 37° C. under 5% CO₂ atmosphere. After this short adjustment period, a layer of mineral oil may be placed on top of each well to maintain CO₂ in the medium.

The plate may then be placed in a spectrophotometer configured to measure the OD at a wavelength of 600 nm for each well at a given time interval for a given total period of time. For example, OD for each well may be measured periodically over a time frame of seconds, minutes, or hours for a period of between 24 and 120 hours. For certain cells measurements for a period of as little as 12 hours may be sufficient. In specific embodiments, measurements may be taken every 5 to 10 minutes. The spectrophotometer may have an incubated chamber to avoid spontaneous death of the cells.

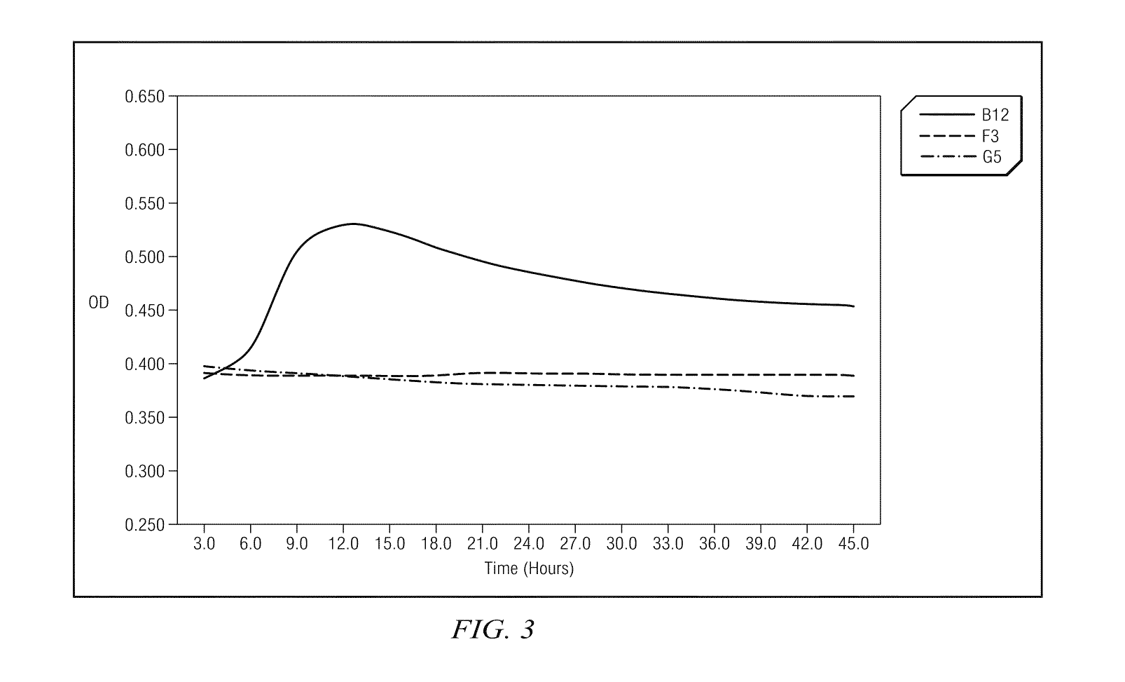

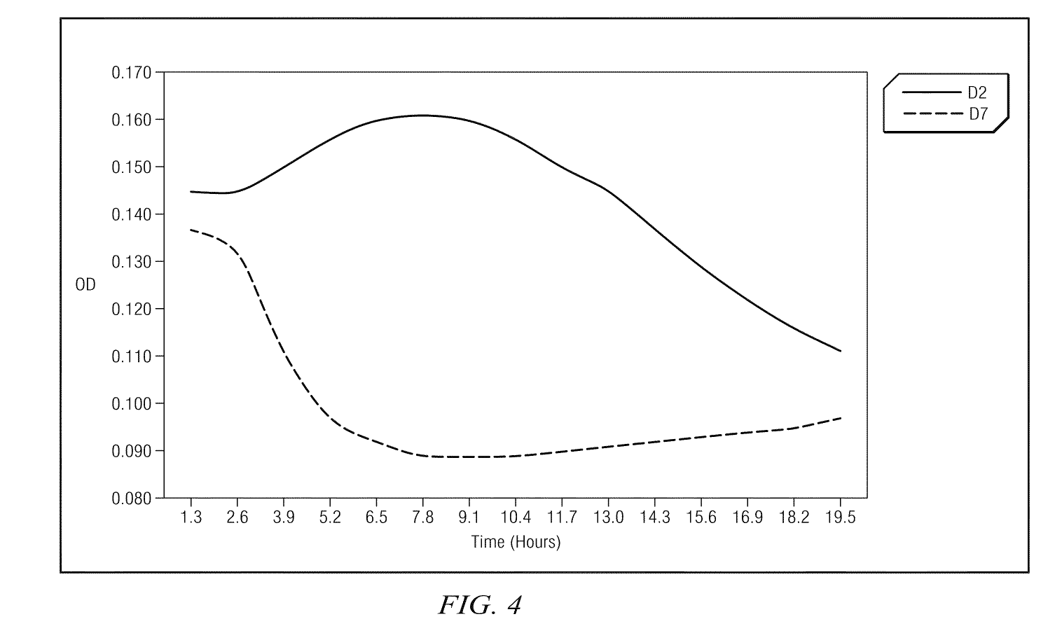

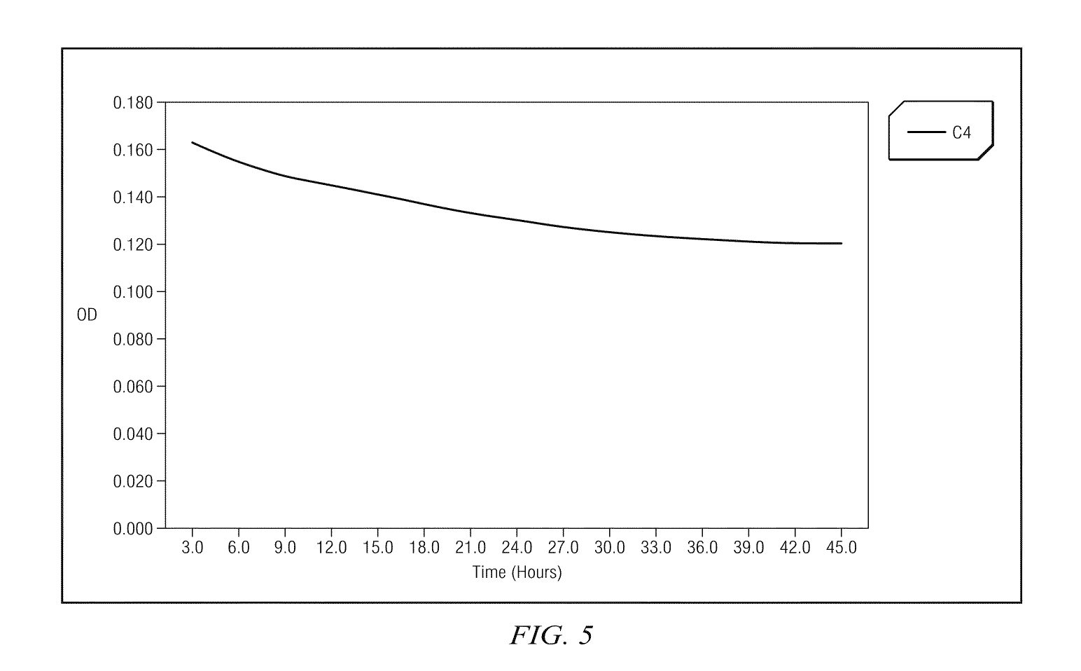

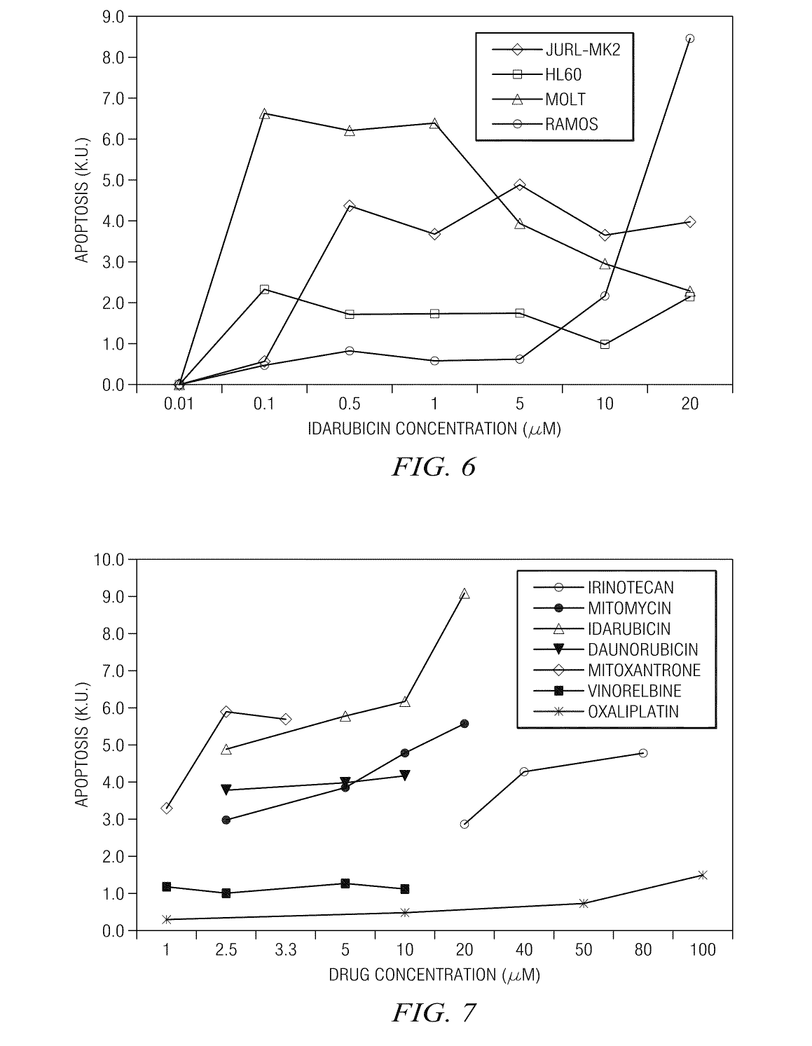

Spectrophotometric data may be converted to kinetic units. Kinetic units are determined by the slope of the curve created when the change in the OD at 600 nm caused by cell blebbing is plotted as a function of time. Specific information regarding the calculation of kinetic units is provided in Kravtsov, Vladimir D. et al., Use of the Microculture Kinetic Assay of Apoptosis to Determine Chemosensitivities of Leukemias, Blood 92:968-980 (1998), incorporated by reference herein. Optical density for a given drug candidate at a given concentration may be plotted against time. This plot gives a distinctive increasing curve if the cells are undergoing apoptosis. An example of the curve obtained when cells undergo apoptosis is shown in FIGS. 3 and 4. In comparison, if the drug candidate has no effect on the cells (e.g. they are resistant), then the curve is similar to that obtained for a control sample with no drug or drug candidate. (FIG. 3.) Cell death due to reasons other than apoptosis can also be determined by the current assay and is useful in eliminating false positives from drug candidate screening. For example, cell necrosis produces a distinctive downward sloping curve easily distinguishable from the apoptosis-related curve as seen in FIG. 4. Further, general cell death also causes a downward curve as seen in FIG. 5.

The effectiveness of a drug candidate may be determined by the value of the kinetic units it produces in a modified MiCK assay using a known cell line. Kinetic units may be determined as follows:

KU=(Vmax_{Drug Treated}−Vmax_{control})×60×y/(OD_{cell}−OD_{blank}).

Vmax is the maximum kinetic rate, which is the slope of the steep increase in the OD v. time plot when cells are undergoing apoptosis. Vmax in this equation is given in milli-optical density units/hour (mOD/h). OD_{cell} is the initial OD of the control containing cells and OD_{blank} is the initial OD of a blank well containing only medium or medium and drug (the drug may be omitted for some drugs, but for colored drugs in particular it may be included in the blank). y is a coefficient dependent on the cell type being assayed and may be determined experimentally through observation of the cell lines. Further information regarding this equation may be found in Kravtsov et al.

In addition to allowing determinations of whether or not a drug candidate causes apoptosis, kinetic unit values generated using the current assay may be compared to determine if a particular drug candidate performs better than or similar to current drugs. Comparison of different concentrations of a drug candidate may also be performed and may give general indications of appropriate dosage. Occasionally some drugs may perform less well at higher concentrations than lower concentrations in some cancers. Comparison of kinetic unit values for different concentrations of drug candidates may identify drug candidates with a similar profile.

Overall, evaluation of an anti-cancer drug candidate may include any determination of the effects of that drug candidate on apoptosis of a cancer cell. Effects may include, but are not limited to induction of apoptosis, degree of induction of apoptosis as compared to known cancer drugs, degree of induction of apoptosis at different drug candidate concentrations, and failure to induce apoptosis. The anti-cancer drug evaluation assay may also be able to detect non-drug-related or non-apoptotic events in the cancer cells, such as cancer cell growth during the assay or cell necrosis.

Any statistically significant positive kinetic unit value may indicate some tendency of a drug candidate to induce apoptosis of a cancer cell. For many clinical purposes, however, drug candidates or concentrations of drugs only able to induce very low levels of apoptosis are not of interest. Accordingly, in certain embodiments of the disclosure, threshold kinetic unit values may be set to distinguish drug candidates able to induce clinically relevant levels of apoptosis in cancer cells. For example, the threshold amount may be 1.5, 2 or 3 kinetic units. The actual threshold selected for a particular drug candidate or concentration of drug candidate may depend on a number of factors. For example, a lower threshold, such as 1.5 or 2, may be acceptable for a drug candidate able to induce apoptosis in cancer types that do not respond to other drugs or respond only to drugs with significant negative side effects. A lower threshold may also be acceptable for drug candidates that exhibit decreased efficacy at higher concentrations or which themselves are likely to have significant negative side effects. A higher threshold, such as 3, may be acceptable for drug candidates able to induce apoptosis in cancer types for which there are already suitable treatments.

### Drug Candidates

According to a specific embodiment, the anti-cancer drug candidates may be any chemicals to be evaluated for the ability to induce apoptosis in cancer cells. These candidates may include various chemical or biological entities such as chemotherapeutics, other small molecules, protein or peptide-based drug candidates, including antibodies or antibody fragments linked to a chemotherapeutic molecule, nucleic acid-based therapies, other biologics, nanoparticle-based candidates, and the like. Drug candidates may be in the same chemical families as existing drugs, or they may be new chemical or biological entities

Drug candidates are not confined to single chemical, biological or other entities. They may include combinations of different chemical or biological entities, for example proposed combination therapies. Further, although many examples herein relate to an assay in which a single drug candidate is applied, assays may also be conducted for multiple drug candidates in combination.

More than one drug candidate, concentration of drug candidate, or combination of drugs or drug candidates may be evaluated in a single assay using a single plate. Different test samples may be placed in different wells. The concentration of the drug candidate tested may be, in particular embodiments, between 0.01 μM and 10,000 μM. The concentration tested may vary by drug type.

### Plate and Spectrophotometer Systems

In specific embodiments, the plate and spectrophotometer may be selected such that the spectrophotometer may read the plate. For example, when using older spectrophotometers, one may use plates with larger wells because the equipment is unable to read smaller-well plates. Newer spectrophotometers may be able to read plate with smaller wells. However, plates with extremely small wells may be avoided due to difficulties in filling the wells, in measuring small volumes accurately, and toxicity of overlying mineral oil, which may increase in small volume wells. In one embodiment, the diameter of the bottom of each well is no smaller than the diameter of the light beam of the spectrophotometer. In a more specific embodiment, the diameter of the bottom of each well is no more than twice the diameter of the light beam of the spectrophotometer. This helps ensure that the OD at 600 nm of a representative portion of the cells in each well is accurately read. The spectrophotometer may make measurement at wavelengths other than 600. For example, the wavelength may be +/−5 or +/−10. However, other wavelengths may be selected so as to be able to distinguish blebbling.

Spectrophotometers may include one or more computers or programs to operate the equipment or to record the results. In one embodiment, the spectrophotometer may be functionally connected to one or more computers able to control the measurement process, record its results, and display or transmit graphs plotting the optical densities as a function of time for each well.

Plates designed for tissue culture may be used, or other plates may be sterilized and treated to make them compatible with tissue culture. Plates that allow cells to congregate in areas not accessible to the spectrophotometer, such as in corners, may work less well than plates that avoid such congregation. Alternatively, more cancer cells may be added to these plates to ensure the presence of a monolayer accessible to the spectrophotometer during the assay. Plates with narrow bottoms, such as the Corning Costar® half area 96 well plate may also assist in encouraging formation of a monolayer at the bottom of the well without requiring inconveniently low sample volumes. Other plates, such as other 96-well plates or smaller well plates, such as 384-well plates, may also be used.

### Cancer Cells

The cancer cells used in the current assay may be any established, well-characterized cancer cell line. Use of an established cell line helps avoid complications, such as mutations of a portion of the cells, that may be difficult to detect and may cause inaccurate test results. In a particular embodiment, the cancer cells may be from any lines commonly used for cancer drug screening in order to obtain FDA or equivalent government approval of a drug to treat a particular cancer.

In general, for accurate results the cancer cell line may be a known cancer cells line, e.g. it may be a monoculture or near monoculture that is generally the same over time, such as a cell line available from the American Type Culture Collection or similar repository. The known cancer cell line may be verifiable as malignant or as having markers used in the art to identify the cell line. For example, the HeLa cell line is a known cervical cancer cell line. Although not required, in some instances a known cancer cell line will be immortalized.

Multiple cancer cell lines may be tested on the same plate in the current assay. However, cell lines with vastly different growth rates or vastly different susceptibilities to control drugs may be tested on different plates due to differences in adjustment and testing times.

During the assay, cancer cells may not always remain as single cell suspensions. For example, solid tumor lines may attach to the surface of the well and form a layer of cells bonded to one another. This attachment and bonding generally may not interfere with the assay, particularly if cells do not overlap or form clumps in a manner that prevents the spectrophotometer measurements from substantially representing the percentage of total cells that undergo blebbing.

### EXAMPLES

The present invention may be better understood through reference to the following examples. These examples are included to describe exemplary embodiments only and should not be interpreted to encompass the entire breadth of the invention.

### Example 1

### Drug Candidate Screening Using Multiple Cell Lines

The drug candidate Idarubicin (4-demethoxydaunorubicin), which is typically used to treat acute myeloid leukemia, was tested for anti-neoplastic, apoptosis-inducing activity against four human leukemia- and lymphoma-derived cell lines using a drug screening assay according to an embodiment of the current disclosure. The cell lines tested were HL60, an acute promyelocytic leukemia cell line, JURL-MK2, a chronic myeloid leukemia in blast crisis, MOLT-3, an acute T-cell lymphoblastic leukemia, and RAMOS, a B cell line derived from Burkitt's lymphoma.

Cancer cells were obtained from exponentially growing cultures in RPMI 1640 medium without phenol red supplemented with 10% heat-inactivated fetal bovine serum, 100 U/ml penicillin and 100 μg/mL streptomycin (complete medium) in humidified air with 5% CO₂ at 37° C. Cells were harvested, washed with pre-warmed medium and resuspended in complete medium. Appropriate cell counts and viability were tested.

Cells from each cell line in complete medium were placed in wells of a 96-well plate at a concentration of 2 to 5×10⁵ cells/ml. Various dilutions of the drug candidate, Idarubicin, were added to the wells in 5 μL aliquots. Final concentrations for the drug candidate were 0.01, 0.1, 0.5, 1, 5, 10 and 20 μM. The plates were incubated at 37° C. for 30 minutes in a humidified 5% CO₂ atmosphere. After this adjustment period, 30 μl of sterile mineral oil was layered on top of each well. The microtiter plate was placed in an incubated spectrophotometer chamber (37° C.) and OD at 600 nm was measured every 5 minutes for a period of 48 hours. The reader was calibrated to zero absorbance using well containing only complete medium and no cells. All tests were performed in triplicate.

Data acquisition and computation was performed using appropriate software. OD readings were plotted against time to provide a kinetic representation of cell responses to the drugs and drug candidate. Kinetic units were calculated for each test well. A kinetic unit below three was considered a negative response and above 3 was considered a positive response. A kinetic unit between 1.5 and 3 was considered a marginal response.

Idarubicin induced apoptotic responses exceeding 3 kinetic units in the MOLT-3, JURL-MK2, and RAMOS cell lines. HL-60 cells had a maximal response to either control drug of 2.3 kinetic units and thus fell slightly short of the threshold for drug sensitivity, exhibiting only marginal sensitivity. Accordingly, the test shows that Idarubicin may not be useful against acute promyelocytic leukemia.

Further, a positive response was seen in the Ramos cell line only at high concentrations, indicating that Idarubicin may have only marginal use in treating Burkitt's lymphoma due to the increased likelihood of side effects at higher drug concentrations.

Finally, the higher kinetic unit readings at lower concentrations of Idarubicin with MOLT-3 cells may indicate that it may be preferable to use lower concentrations of Idabubicin to treat acute T-cell lymphoblastic leukemia.

### Example 2

### Screening Using Multiple Drug Candidates or Chemotherapy Agents on a Single Cell Line

A variety of anti-cancer drugs were tested against a single ovarian cancer cell line, CAOV-3, to determine their suitability for use against ovarian cancer. Tests were performed in a manner similar to those in Example 1, but with higher drug concentrations. Results show that Vinorelbine and Oxaliplatin are not suitable drugs for treating ovarian cancer. Results also show that Irinotecan may have only marginal use in treating ovarian cancer due to the need to use high concentrations of the drug to achieve a positive response. Mitomycin, Idarubicin, Daunorubicin, and Mitoxantrone all demonstrate a positive response at reasonable concentrations and thus are suitable drug candidates for treatment of ovarian cancer.

Although only exemplary embodiments of the invention are specifically described above, it will be appreciated that modifications and variations of these examples are possible without departing from the spirit and intended scope of the invention. For example, in the specification particular measurements are given. It would be understood by one of ordinary skill in the art that in many instances other values similar to, but not exactly the same as the given measurements may be equivalent and may also be encompassed by the present invention.

1. A method of evaluating the ability of an anti-cancer drug candidate to induce apoptosis in a known cancer cell line comprising:
placing a single-cell suspension of viable cancer cells from a known cancer cell line in at least one well of a plate able to be read by a spectrophotometer, wherein the cancer cells are in a concentration sufficient to form a monolayer of cells on a bottom of the well;
adding at least one drug candidate to the well in an amount sufficient to achieve a target drug candidate concentration;
measuring the optical density of the well at a wavelength of approximately 600 nm using a spectrophotometer at selected time intervals for a selected duration of time;
determining a kinetic units value from the optical density and time measurements;
correlating the kinetic units value with:
a) an ability of the anti-cancer drug candidate to induce apoptosis in the cancer cell line if the kinetic units value is positive;
b) an inability of the anti-cancer drug candidate to induce apoptosis in the cancer cell line if the kinetic units value is not positive.

2. The method according to claim 1, wherein correlating comprises correlating the kinetic units value with:
a) an ability of the anti-cancer drug candidate to induce apoptosis in the cancer cell line if the kinetic units value is greater than 1.5;
b) an inability of the anti-cancer drug candidate to induce apoptosis in the cancer cell line if the kinetic units value is less than 1.5.

3. The method according to claim 1, wherein correlating comprises correlating the slope of the approximately linear region with:
a) an ability of the anti-cancer drug candidate to induce apoptosis in the cancer cell line if the kinetic units value is greater than 2;
b) an inability of the anti-cancer drug candidate to induce apoptosis in the cancer cell line if the kinetic units value is less than 2.

4. The method according to claim 1, wherein correlating comprises correlating the slope of the approximately linear region with:
a) an ability of the anti-cancer drug candidate to induce apoptosis in the cancer cell line if the kinetic units value is greater than 3;
b) an inability of the anti-cancer drug candidate to induce apoptosis in the cancer cell line if the kinetic units value is less than 3.

5. The method according to claim 1, wherein correlating comprises correlating the kinetic units value with induction of spontaneous cell death or necrosis in the cancer cell line by the anti-cancer drug candidate if the slope of a plot of the optical density over the duration of time is negative.

6. The method according to claim 1, wherein the cancer cells are in a concentration of between 2×10⁵ and 1×10⁶ cells/mL.

7. The method according to claim 1, wherein the cancer cells are in an exponential growth phase.

8. The method according to claim 1, wherein cancer cells are placed in multiple wells of the plate and each well has a different target drug candidate concentration.

9. The method according to claim 1, comprising adding at least one additional drug candidate to the well in an amount sufficient to achieve an additional target drug candidate concentration.

10. The method according to claim 1, wherein the target drug candidate concentration is between 0.01 and 10,000 μM.

11. The method according to claim 1, wherein the selected time intervals are 5 to 10 minutes.

12. The method according to claim 1, wherein the duration of time is between 12 hours and 120 hours.

13. A method of evaluating the ability of an anti-cancer drug candidate to induce apoptosis in a cancer cell type comprising:
placing a single-cell suspension of viable cancer cells from a known cancer cell line in at least one well of a plate able to be read by a spectrophotometer, wherein the cancer cells are in a concentration sufficient to form a monolayer of cells on a bottom of the well, and wherein the known cancer cell line is of the cancer cell type;
adding a drug candidate to the well in an amount sufficient to achieve a target drug candidate concentration;
measuring the optical density of the well at a wavelength of approximately 600 nm using a spectrophotometer at selected time intervals for a selected duration of time;
determining a kinetic units value from the optical density and time measurements;
correlating the kinetic units value with:
a) an ability of the anti-cancer drug candidate to induce apoptosis in the cancer cell type if the kinetic units value is positive;
b) an inability of the anti-cancer drug candidate to induce apoptosis in the cancer cell type if the kinetic units value is not positive.

14. The method according to claim 13, wherein correlating comprises correlating the kinetic units value with:
a) an ability of the anti-cancer drug candidate to induce apoptosis in the cancer cell type if the kinetic units value is greater than 1.5;
b) an inability of the anti-cancer drug candidate to induce apoptosis in the cancer cell type if the kinetic units value is less than 1.5.

15. The method according to claim 13, wherein correlating comprises correlating the slope of the approximately linear region with:
a) an ability of the anti-cancer drug candidate to induce apoptosis in the cancer cell type if the kinetic units value is greater than 2;
b) an inability of the anti-cancer drug candidate to induce apoptosis in the cancer cell type if the kinetic units value is less than 2.

16. The method according to claim 13, wherein correlating comprises correlating the slope of the approximately linear region with:
a) an ability of the anti-cancer drug candidate to induce apoptosis in the cancer cell type if the kinetic units value is greater than 3;
b) an inability of the anti-cancer drug candidate to induce apoptosis in the cancer cell type if the kinetic units value is less than 3.

17. The method according to claim 13, wherein correlating comprises correlating the kinetic units value with induction of spontaneous cell death or necrosis in the cancer cell type by the anti-cancer drug candidate if the slope of a plot of the optical density over the duration of time is negative.

18. The method according to claim 13, wherein the cancer cells are in a concentration of between 2×10⁵ and 1×10⁶ cells/mL.

19. The method according to claim 13, wherein the cancer cells are in an exponential growth phase.

20. The method according to claim 13, wherein cancer cells are placed in multiple wells of the plate and each well has a different target drug candidate concentration.

21. The method according to claim 13, comprising adding at least one additional drug candidate to the well in an amount sufficient to achieve an additional target drug candidate concentration.

22. The method according to claim 13, wherein the target drug candidate concentration is between 0.01 and 10,000 μM.

23. The method according to claim 13, wherein the selected time intervals are 5, 15, 30, or 60 minutes.

24. The method according to claim 13, wherein the duration of time is between 12 hours and 120 hours.
